(19) United States
(12) Patent Application Publication
(10) Pub. No.: US 2016/0008794 A1
(43) Pub. Date: Jan. 14, 2016

(54) CATALYST FOR ACRYLONITRILE PRODUCTION AND METHOD FOR PRODUCING ACRYLONITRILE

(71) Applicant: MITSUBISHI RAYON CO., LTD., Chiyoda-ku, Tokyo (JP)

(72) Inventors: Kazufumi NISHIDA, Yokohama-shi (JP); Motoo YANAGITA, Yokohama-shi (JP); Takashi KARASUDA, Yokohama-shi (JP); Hirokazu WATANABE, Otake-shi (JP)

(73) Assignee: Mitsubishi Rayon Co., Ltd., Chiyoda-ku, Tokyo (JP)

(21) Appl. No.: 14/768,281

(22) PCT Filed: Feb. 19, 2014
(86) PCT No.: PCT/JP2014/053902

§ 371 Date: Aug. 17, 2015

(30) Foreign Application Priority Data
Feb. 21, 2013 (JP) 2013-032047

(51) Int. Cl. B01J 27/19; C07C 253/26; B01J 37/08

(52) U.S. Cl. CPC B01J 27/19; B01J 37/08; C07C 253/26

(57) ABSTRACT

A catalyst for acrylonitrile production, which is produced by the vapor phase contact ammoxidation of propylene by molecular oxygen and ammonia, and a method for producing acrylonitrile using the catalyst.

### TECHNICAL FIELD

The present invention relates to a catalyst for acrylonitrile production, which is produced by the vapor phase contact ammoxidation of propylene by molecular oxygen and ammonia, and a method for producing acrylonitrile using the catalyst.

The present application claims the priority based on Japanese Patent Application No. 2013-032047 filed on Feb. 21, 2013 in Japan, and the disclosure content thereof is incorporated herein by reference.

### BACKGROUND ART

As a method for producing acrylonitrile, there is widely known a method for the vapor phase contact ammoxidation of propylene by molecular oxygen and ammonia in the presence of a catalyst. As the catalyst used at this time, there are disclosed various catalysts until now. For example, Patent Document 1 discloses a complex oxide catalyst including antimony and at least one kind of elements selected from the group consisting of iron, cobalt, and nickel. Patent Documents 2 to 9 disclose a complex oxide catalyst including iron, antimony, tellurium, vanadium, molybdenum, tungsten, and the like. Patent Documents 10 to 12 disclose a method for preparing a catalyst including iron and antimony.

In addition, Patent Documents 13 to 20 disclose a complex oxide catalyst including molybdenum, bismuth, iron, and the like.

### CITATION LIST

### Patent Document

Patent Document 1: JP 38-19111 B

Patent Document 2: JP 46-2804 B

Patent Document 3: JP 47-19765 B

Patent Document 4: JP 47-19766 B

Patent Document 5: JP 47-19767 B

Patent Document 6: JP 50-108219 A

Patent Document 7: JP 52-125124 A

Patent Document 8: JP 4-118051 A

Patent Document 9: JP 5011176 B1

Patent Document 10: JP 47-18722 B

Patent Document 11: JP 47-18723 B

Patent Document 12: JP 59-139938 A

Patent Document 13; JP 38-17967 B

Patent Document 14: JP 59-204163 A

Patent Document 15: JP 61-13701 B

Patent Document 16: JP 1-228950 A

Patent Document 17: JP 3534431 B1

Patent Document 18: JP 10-043595 A

Patent Document 19: JP 11-169715 A

Patent Document 20: JP 2001-114740 A

### SUMMARY OF THE INVENTION

### Problem to be Solved by the Invention

However, these catalysts are yet insufficient in terms of the yield of acrylonitrile, and it is required to further improve the catalyst from the industrial view.

The invention is made with consideration for the above-described circumstances, and an object of the invention is to provide a catalyst for acrylonitrile production and a method for producing acrylonitrile, which are capable of producing acrylonitrile in high yield as compared with the conventional catalysts.

### Means for Solving Problem

The present inventors enthusiastically reviewed the catalyst for acrylonitrile production, which includes iron, antimony, and tellurium, and as a result, found that a catalyst with high yield of acrylonitrile can be obtained by further combining specific components to these components in specific ratios. Accordingly, the present inventors completed the invention.

In other words, a catalyst for acrylonitrile production according to the invention is characterized in that the catalyst has the compositions represented by the following General Formula:

FeₐSb_{b}C_{c}D_{d}TeₑCo_{f}G_{g}XₓY_{y}Z_{z}Oₕ(SiO₂)ᵢ

In Formula, Fe is iron; Sb is antimony; Te is tellurium; Co is cobalt; C is at least one kind of elements selected from the group consisting of copper and nickel; D is at least one kind of elements selected from the group consisting of molybdenum, tungsten, and vanadium; G is at least one kind of elements selected from the group consisting of phosphorous and boron; X is at least one kind of elements selected from the group consisting of tin, titanium, zirconium, niobium, tantalum, ruthenium, palladium, silver, aluminum, gallium, indium, thallium, germanium, arsenic, bismuth, lanthanum, cerium, praseodymium, neodymium, and samarium; Y is at least one kind of elements selected from the group consisting of magnesium, calcium, strontium, barium, manganese, zinc, and lead; Z is at least one kind of elements selected from the group consisting of lithium, sodium, potassium, rubidium, and cesium; O is oxygen; (SiO₂) represents silica; a, b, c, d, e, f, g, x, y, z, h, and i represent the atomic ratios of respective elements (in the case of silica, atomic ratio of silicon); a is 10; b is 5 to 60; c is 1 to 8; d is 0.1 to 4; e is 0.1 to 5; f is 0.1 to 4.5; g is 0.1 to 5; x is 0 to 5; y is 0 to 5; z is 0 to 2; i is 10 to 200; h is the atomic ratio of oxygen that is necessary to meet the atomic values of represent elements except silicon; and (a+f)/b is 0.50 or more and 0.60 or less.

In addition, the catalyst for acrylonitrile production according to the invention preferably includes iron antimonate in a crystal phase.

In addition, the method for producing acrylonitrile according to the invention is characterized in that the method produces acrylonitrile by reacting propylene with molecular oxygen and ammonia in the presence of the catalyst for acrylonitrile production according to the invention.

In other words, the invention has the following aspects.

[1] A catalyst for acrylonitrile production, the catalyst having the composition represented by the following General Formula:

FeₐSb_{b}C_{c}D_{d}TeₑCo_{f}G_{g}XₓY_{y}Z_{z}Oₕ(SiO₂)ᵢ

(In Formula, Fe is iron; Sb is antimony; Te is tellurium; Co is cobalt;

C is at least one kind of elements selected from the group consisting of copper and nickel;

D is at least one kind of elements selected from the group consisting of molybdenum, tungsten, and vanadium;

G is at least one kind of elements selected from the group consisting of phosphorous and boron;

X is at least one kind of elements selected from the group consisting of tin, titanium, zirconium, niobium, tantalum, ruthenium, palladium, silver, aluminum, gallium, indium, thallium, germanium, arsenic, bismuth, lanthanum, cerium, praseodymium, neodymium, and samarium;

Y is at least one kind of elements selected from the group consisting of magnesium, calcium, strontium, barium, manganese, zinc, and lead;

Z is at least one kind of elements selected from the group consisting of lithium, sodium, potassium, rubidium, and cesium;

O is oxygen;

(SiO₂) represents silica;

a, b, c, d, e, f, g, x, y, z, h, and i represent the atomic ratios of respective elements (in the case of silica, atomic ratio of silicon);

a is 10; b is 5 to 60; c is 1 to 8; d is 0.1 to 4; e is 0.1 to 5; f is 0.1 to 4.5; g is 0.1 to 5; x is 0 to 5; y is 0 to 5; z is 0 to 2; i is 10 to 200; h is the atomic ratio of oxygen that is necessary to meet the atomic values of represent elements except silicon; and

(a+1)/b is 0.50 or more and 0.60 or less.)

[2] The catalyst for acrylonitrile production disclosed in [1], the catalyst including iron antimonate in a crystal phase.

[3] A method for producing acrylonitrile, the method including reacting propylene with molecular oxygen and ammonia in the presence of the catalyst for acrylonitrile production disclosed in [1] or [2].

### Effect of the Invention

According to the catalyst for acrylonitrile production of the invention, it is possible to produce acrylonitrile in high yield by suppressing the production of by-product as compared with the conventional catalysts.

### MODE(S) FOR CARRYING OUT THE INVENTION

Hereinafter, the invention will be described in detail.

As one embodiment of the invention, there is a catalyst for acrylonitrile production, in which the catalyst has the composition represented by the following General Formula:

FeₐSb_{b}C_{c}D_{d}TeₑCo_{f}G_{g}XₓY_{y}Z_{z}Oₕ(SiO₂)ᵢ

In Formula, each of the symbols is as follows:

Fe is iron;

Sb is antimony;

Te is tellurium;

Co is cobalt;

C is at least one kind of elements selected from the group consisting of copper and nickel;

D is at least one kind of elements selected from the group consisting of molybdenum, tungsten, and vanadium;

G is at least one kind of elements selected from the group consisting of phosphorous and boron;

X is at least one kind of elements selected from the group consisting of tin, titanium, zirconium, niobium, tantalum, ruthenium, palladium, silver, aluminum, gallium, indium, thallium, germanium, arsenic, bismuth, lanthanum, cerium, praseodymium, neodymium, and samarium;

Y is at least one kind of elements selected from the group consisting of magnesium, calcium, strontium, barium, manganese, zinc, and lead;

Z is at least one kind of elements selected from the group consisting of lithium, sodium, potassium, rubidium, and cesium;

O is oxygen; and

(SiO₂) represents silica.

In addition, in Formula, a, b, c, d, f, e, g, x, y, z, h, and i represent the atomic ratios of respective elements (in the case of silica, atomic ratio of silicon);

a is 10;

b is 5 to 60, and preferably 10 to 55; c is 1 to 8, and preferably 1.5 to 7.5; d is 0.1 to 4, and preferably 0.3 to 3;

e is 0.1 to 5, and preferably 0.5 to 4.5;

f is 0.1 to 4.5, and preferably 0.2 to 3.5; g is 0.1 to 5, and preferably 0.3 to 4; x is 0 to 5, and preferably 0 to 4.5; y is 0 to 5, and preferably 0 to 4.5; z is 0 to 2, and preferably 0 to 1.5; i is 10 to 200, and preferably 20 to 180; h is the atomic ratio of oxygen that is necessary to meet the atomic values of represent elements except silicon; and (a+f)/b is 0.50 or more and 0.60 or less, the lower value thereof is preferably 0.52, and the upper value thereof is preferably 0.57.

When the atomic ratios of represent elements that is included in the catalyst are out of the above-described ranges, the yield of acrylonitrile decreases. Therefore, the effect of the invention is not sufficiently exhibited, and it is difficult to achieve the purpose of the invention.

In the invention, the composition of the catalyst for acrylonitrile production indicates the bulk composition of a catalyst, but unless the component with significantly high volatility is used, the composition of the catalyst (atomic ratio) may be calculated from the added amount of raw materials that are used for respective elements constituting the catalyst.

In other words, in the invention, the composition of the catalyst for acrylonitrile production may be the composition (atomic ratio) that is calculated from the added amounts of raw materials of represent elements constituting the catalyst.

In addition, in one embodiment of the invention, the catalyst preferably includes iron antimonate in a crystal phase. There are various kinds of compositions of iron antimonate (see Patent Document 8 described above), but FeSbO₄ is the most popular. In the invention, it may be considered that the composition of iron antimonate is mostly FeSbO₄. Whether or not the crystal phase of iron antimonate is existed may be confirmed by X-ray diffraction. In the invention, iron antimonate in which various elements is solid-dissolved may be used in addition to pure iron antimonate.

In addition, all of Fe components and Sb components do not always form iron antimonate in a crystal phase. Some Fe components or Sb components may be existed in a free state, or may be formed in any kinds of other compounds.

According to one embodiment of the invention, the catalyst for acrylonitrile production includes iron antimonate in a crystal phase, and thus, it is possible to improve the activity of the catalyst and also to make the physical properties such as particle strength and bulk density preferable.

A method for preparing the catalyst for acrylonitrile production according to the invention is not particularly limited. However, a method including preparing an aqueous slurry including raw materials for respective elements constituting a catalyst, drying the aqueous slurry obtained, and calcining the dried product is preferable.

In other words, as a method for preparing the catalyst for acrylonitrile production according to the invention, there may be a method including preparing an aqueous slurry including raw materials for respective elements constituting the catalyst, drying the aqueous slurry obtained, and calcining the dried product.

The aqueous slurry may include all of the elements that are desired to constitute a catalyst in the desired atomic ratios, or some elements may be added by a method such as impregnation in the composition after drying or calcining and some elements may be adjusted to be desired atomic ratios, and then may be calcined.

In addition, in the case of producing a catalyst including iron antimonate in a crystal phase, for example, the method disclosed in Patent Document 10 or Patent Document 11 may be used.

In other words, the catalyst including iron antimonate in a crystal phase can be prepared by the method including preparing the aqueous slurry including antimony raw materials, a trivalent iron compound, and nitrite ion, adjusting the pH of the slurry to be 7 or less, heat-treating the slurry at the temperature range of 40 to 150° C., drying the obtained slurry, and then calcining the dried product.

In other words, as a method for producing a catalyst including iron antimonate in a crystal phase, there may be the preparing methods, such as the method including preparing the aqueous slurry including antimony raw materials, a trivalent iron compound, and nitrite ion, adjusting the pH of the slurry to be 7 or less, heat-treating the slurry at the temperature range of 40 to 150° C., drying the obtained slurry, and then calcining the dried product, and the method including drying or calcining the above-described aqueous slurry, adding the rest elements by a method such as impregnation, and adjusting to be desired atomic ratios.

In addition, the preparing method may include calcining after adjusting the slurry to be desired atomic ratios.

The raw materials for respective elements are not particularly limited. The oxides of respective elements, and nitrate, carbonate, organic acid salts, ammonium salts, hydroxides, and halides, which are capable of easily becoming to be oxides by heating, may be used. In addition, they may be used in combination of two or more.

For example, the raw materials for iron components are not particularly limited as long as they can be easily converted into oxides.

In addition, in the case of preparing a catalyst including iron antimonate in a crystal phase, iron may be preferably existed as a trivalent ion in a solution or slurry, and for example, the raw materials prepared by dissolving inorganic acid salts such as ferric nitrate and ferric sulfate; organic acid salts such as iron citrate; and metal irons such as electrolytic iron powder, in nitric acid may be preferably used.

The antimony components are not particularly limited, and oxides such as antimony trioxide or antimony pentoxide, antimony chloride, antimony sulfate, and the like, can be used.

The raw materials for tellurium components are not particularly limited, and in addition to tellurium dioxide and telluric acid, the solution prepared by dissolving metal tellurium in nitric acid or hydrogen peroxide solution can be used.

The raw materials for cobalt components are not particularly limited, and oxides such as cobalt oxide, chlorides such as cobalt chloride, cobalt nitrate, and the like can be used.

The raw materials for silica are not particularly limited, and colloidal silica may be preferably used. The colloidal silica prepared by the known method may be used, and the colloidal silica available on the market may be properly selected and used.

The size of colloid particle in the colloidal silica is not particularly limited, and the average diameter thereof is preferably 2 to 100 nm and more preferably 5 to 75 nm. The colloidal silica may be the colloidal silica having uniform colloid particle size, or may be the colloidal silica having the colloid particles with various types of size. In addition, a plurality of colloidal silica with different average diameters and different pHs may be mixed and used.

A method for drying the aqueous slurry is not particularly limited, and may be arbitrarily selected from the known methods and used.

In one embodiment of the invention, a catalyst may be applied for both a fixed bed reactor and fluidized bed reactor. In other words, in one embodiment of the invention, a catalyst can be used as a fixed bed catalyst or a fluidized bed catalyst, but may be preferably used as a fluidized bed catalyst, in particular.

In one embodiment of the invention, in the case of using the catalyst for acrylonitrile production as a fluidized bed catalyst, it is preferable to obtain the particles dried by using a spray dryer. The above particles preferably have a globular shape. As the spray drier, the known spray drier such as a rotary disc type or nozzle type drier can be used. When performing a spray drying, the conditions for the spray drying may be properly adjusted so as to obtain the catalyst having the physical properties that are preferred as a fluidized bed catalyst, for example, particle size distribution, particle strength, and the like.

In addition, in one embodiment of the invention, when the catalyst for acrylonitrile production is used for a fluidized bed, it is preferably the granular material having the outer diameter thereof in the range of 1 to 200 μm, and more preferably in the range of 5 to 150 μm. The shape of the granular material is preferably a granular shape.

By calcining the obtained dried product at the temperature range of 550 to 1000° C., the preferred catalyst structure is formed, and thus the activity as a catalyst is exhibited. The calcination time is not particularly limited, but when it is too short, it is difficult to obtain a favorable catalyst, and thereby, 0.5 hour or longer is preferable and 1 hour or longer is more preferable. The upper limit thereof is not particularly limited, but even when the calcination is performed for a long period of time that is longer than needs, the effect over a certain level is not obtained, and thus it is generally within 20 hours.

The calcination method is not particularly limited, and general-purpose calcining furnaces can be used. When preparing a fluidized bed catalyst, a rotary kiln, a fluidized bed furnace, and the like are particularly preferably used.

On calcining, the dried product may be immediately calcined at the temperature range of 550 to 1000° C., but by performing the calcination at the temperature range of 550 to 1000° C. after pre-calcining in one to two steps at the temperature range of 250 to 500° C., the physical properties and activity of the catalyst may be improved in some cases.

In one embodiment of the invention, when acrylonitrile is prepared by the vapor phase contact ammoxidation of propylene by molecular oxygen (O₂, hereinafter, simply referred to as oxygen) and ammonia using the catalyst for acrylonitrile production, a fluidized bed reactor may be preferably used.

The concentration of propylene in raw material gas when performing the vapor phase contact ammoxidation reaction may be changed within the wide range, and 1 to 20 vol % is proper and 3 to 15 vol % is particularly preferable.

The mole ratio of propylene and oxygen (propylene:oxygen) in raw material gas is preferably 1:1.5 to 1:3. Air is used industrially advantageously as an oxygen source, but if necessary, by adding pure oxygen, the oxygen enriched air may be used.

In addition, the mole ratio of propylene and ammonia (propylene:ammonia) in reaction gas is preferably 1:1 to 1:1.5.

The raw material gas may be diluted with inert gas or water vapor.

The vapor phase contact ammoxidation reaction is generally performed at the reaction temperature of 370 to 500° C., the reaction pressure of normal pressure to 500 kPa, and the apparent contact time between a catalyst and raw material gas of 1 to 20 seconds.

In addition, in the invention, “apparent contact time” is the value obtained by the following Equation.

Apparent contact time (sec)=catalyst volume based on apparent bulk density (mL)/raw material gas amount converted by reaction condition (mL/sec)

### EXAMPLES

Hereinafter, the effect of the invention will be described in detail with reference to Examples and Comparative Examples, but the invention is not limited to the following Examples.

### Example 1

### Preparation of Catalyst

The catalyst having the composition listed in Table 1 was prepared by the following procedures.

42.7 parts by mass of a copper powder was dissolved in 1800 parts by mass of 63% by mass of nitric acid. To the obtained solution, 1750 parts by mass of pure water was added, and then heated to be 60° C. 150 parts by mass of an electrolytic iron powder and 34.3 parts by mass of a tellurium powder were added little by little, and then dissolved. After confirming the dissolution, to the above solution, 39.1 parts by mass of cobalt nitrate, 39.1 parts by mass of nickel nitrate, and 6.3 parts by mass of calcium nitrate were sequentially added and then dissolved (Solution A).

Separately, the solution (Solution B) was prepared by dissolving 35.1 parts by mass of ammonium paratungstate in 1750 parts by mass of pure water and the solution (Solution C) was prepared by dissolving 47.4 parts by mass of ammonium paramolybdate and 34.3 parts by mass of a tellurium powder in 250 parts by mass of pure water and 100 parts by mass of 35% by mass of a hydrogen peroxide solution.

While stirring, 4437 parts by mass of 20% by mass of colloidal silica, 743.8 parts by mass of antimony trioxide powder, Solution B, and Solution C were sequentially added to Solution A to obtain the aqueous slurry.

To the aqueous slurry, 15% by mass of ammonia water was dropped to adjust the pH thereof to be 2.0. The aqueous slurry thus obtained was heat-treated at the boiling point for 3 hours under reflux.

The aqueous slurry after heat-treating was cooled to be 80° C., and then 6.2 parts by mass of 85% by mass of phosphoric acid, 33.2 parts by mass of boric acid, and 1.0 part by mass of lithium nitrate were sequentially added thereto.

The obtained aqueous slurry was spray-dried under the temperature of drying air, that is, 330° C. at the inlet of a drier and 160° C. at the outlet of a drier by a spray drier to obtain the dried particles having a globular shape. Then, the obtained dried particles were calcined at 250° C. for 2 hours and at 400° C. for 2 hours, and finally were calcined at 795° C. for 3 hours using a fluidized bed furnace to obtain a catalyst including iron antimonate in a crystal phase.

(Catalyst Performance Test)

Using the obtained catalyst, the reaction for producing acrylonitrile by the vapor phase contact ammoxidation reaction of propylene was performed in the following manner.

The catalyst was filled in a fluidized bed reactor, in which the inner diameter of the catalytic flowing part was 55 mm and the height thereof was 2000 mm, to be the apparent contact time of the catalyst and raw material gas as listed in Table 2.

The raw material gas having the composition of propylene:ammonia:oxygen=1:1.1:2.3 (mole ratio) using air as an oxygen source was entered in the catalyst bed at the gas line rate of 17 cm/sec. The reaction pressure was 200 kPa and the reaction temperature was 460° C.

For the quantification of reaction products, a gas chromatography was performed to obtain the propylene conversion rate and acrylonitrile yield at 4 hours after initiating the reaction. At this time, the propylene conversion rate and acrylonitrile yield were obtained by the following Equations.

Propylene conversion rate (%)=(molar number of reaction−consumed propylene/molar number of propylene supplied as raw material gas)×100

Acrylonitrile yield (%)=(molar number of produced acrylonitrile/molar number of propylene supplied as raw material gas)×100

### Example 2

The catalyst was prepared in the same procedures as in Example 1, except that the added amount of cobalt nitrate and the added amount of an antimony trioxide powder were changed into 140.7 parts by mass and 861.2 parts by mass, respectively, in Example 1.

For the obtained catalyst, the catalyst performance test was performed in the same method as in Example 1. The results thus obtained are listed in Table 2.

### Example 3

The catalyst was prepared in the same procedures as in Example 1, except that the added amount of cobalt nitrate and the added amount of an antimony trioxide powder were changed into 109.4 parts by mass and 783.0 parts by mass, respectively, in Example 1.

For the obtained catalyst, the catalyst performance test was performed in the same method as in Example 1. The results thus obtained are listed in Table 2.

### Example 4

The catalyst having the composition listed in Table 1 was prepared by the following procedures.

34.1 parts by mass of a copper powder was dissolved in 1800 parts by mass of 63% by mass of nitric acid. To the obtained solution, 1750 parts by mass of pure water was added, and then heated to be 60° C. 150 parts by mass of an electrolytic iron powder and 51.4 parts by mass of a tellurium powder were added little by little, and then dissolved. After confirming the dissolution, to the above solution, 117.2 parts by mass of cobalt nitrate, 93.7 parts by mass of nickel nitrate, and 6.4 parts by mass of indium nitrate were sequentially added and then dissolved (Solution A).

Separately, the solution (Solution B) was prepared by dissolving 14.0 parts by mass of ammonium paratungstate in 700 parts by mass of pure water and the solution (Solution C) was prepared by dissolving 71.1 parts by mass of ammonium paramolybdate and 51.4 parts by mass of a tellurium powder in 400 parts by mass of pure water and 150 parts by mass of 35% by mass of a hydrogen peroxide solution.

While stirring, 4679 parts by mass of 20% by mass of colloidal silica, 822.1 parts by mass of an antimony trioxide powder, Solution B, and Solution C were sequentially added to Solution A to obtain the aqueous slurry.

To the aqueous slurry, 15% by mass of ammonia water was dropped to adjust the pH thereof to be 2.0. The aqueous slurry thus obtained was heat-treated at the boiling point for 3 hours under reflux.

The aqueous slurry after heat-treating was cooled to be 80° C., and then 31.0 parts by mass of 85% by mass of phosphoric acid, 16.6 parts by mass of boric acid, and 2.7 parts by mass of potassium nitrate were sequentially added thereto.

The obtained aqueous slurry was spray-dried under the temperature of drying air, that is, 330° C. at the inlet of a drier and 160° C. at the outlet of a drier by a spray drier to obtain the dried particles having a globular shape. Then, the obtained dried particles were calcined at 250° C. for 2 hours and at 400° C. for 2 hours, and finally were calcined at 785° C. for 3 hours using a fluidized bed furnace to obtain a catalyst including iron antimonate in a crystal phase.

For the obtained catalyst, the catalyst performance test was performed in the same method as in Example 1. The results thus obtained are listed in Table 2.

### Example 5

The catalyst was prepared in the same procedures as in Example 4, except that the added amount of cobalt nitrate was changed into 93.8 parts by mass in Example 4.

For the obtained catalyst, the catalyst performance test was performed in the same method as in Example 1. The results thus obtained are listed in Table 2.

### Example 6

The catalyst having the composition listed in Table 1 was prepared by the following procedures.

25.6 parts by mass of a copper powder was dissolved in 1750 parts by mass of 63% by mass of nitric acid. To the obtained solution, 1700 parts by mass of pure water was added, and then heated to be 60° C. 150 parts by mass of an electrolytic iron powder and 68.5 parts by mass of a tellurium powder were added little by little, and then dissolved. After confirming the dissolution, to the above solution, 195.4 parts by mass of cobalt nitrate, 156.2 parts by mass of nickel nitrate, 11.7 parts by mass of praseodymium nitrate, and 4.5 parts by mass of lead nitrate were sequentially added, and then dissolved (Solution A).

Separately, the solution (Solution B) was prepared by dissolving 14.0 parts by mass of ammonium paratungstate in 700 parts by mass of pure water and the solution (Solution C) was prepared by dissolving 56.9 parts by mass of a ammonium paramolybdate and 68.5 parts by mass of a tellurium powder in 400 parts by mass of pure water and 250 parts by mass of 35% by mass of a hydrogen peroxide solution.

While stirring, 4841 parts by mass of 20% by mass of colloidal silica, 861.3 parts by mass of an antimony trioxide powder, Solution B, and Solution C were sequentially added to Solution A to obtain the aqueous slurry.

To the aqueous slurry, 15% by mass of ammonia water was dropped to adjust the pH thereof to be 2.0. The aqueous slurry thus obtained was heat-treated at the boiling point for 3 hours under reflux.

The aqueous slurry after heat-treating was cooled to be 80° C., and then 31.0 parts by mass of 85% by mass of phosphoric acid was added thereto.

The obtained aqueous slurry was spray-dried under the temperature of drying air, that is, 330° C. at the inlet of a drier and 160° C. at the outlet of a drier by a spray drier to obtain the dried particles having a globular shape. Then, the obtained dried particles were calcined at 250° C. for 2 hours and at 400° C. for 2 hours, and finally were calcined at 780° C. for 3 hours using a fluidized bed furnace to obtain a catalyst including iron antimonate in a crystal phase.

For the obtained catalyst, the catalyst performance test was performed in the same method as in Example 1. The results thus obtained are listed in Table 2.

### Example 7

The catalyst was prepared in the same procedures as in Example 6, except that the added amount of an antimony trioxide powder was changed into 978.8 parts by mass in Example 6.

For the obtained catalyst, the catalyst performance test was performed in the same method as in Example 1. The results thus obtained are listed in Table 2.

### Example 8

The catalyst was prepared in the same procedures as in Example 6, except that the added amount of an antimony trioxide powder was changed into 822.2 parts by mass in Example 6.

For the obtained catalyst, the catalyst performance test was performed in the same method as in Example 1. The results thus obtained are listed in Table 2.

### Comparative Example 1

The catalyst was prepared in the same procedures as in Example 1, except that the added amount of an antimony trioxide powder was changed into 861.2 parts by mass in Example 1.

For the obtained catalyst, the catalyst performance test was performed in the same method as in Example 1. The results thus obtained are listed in Table 2.

### Comparative Example 2

The catalyst was prepared in the same procedures as in Example 1, except that the added amount of cobalt nitrate and the added amount of an antimony trioxide powder were changed into 156.3 parts by mass and 743.8 parts by mass, respectively, in Example 1.

For the obtained catalyst, the catalyst performance test was performed in the same method as in Example 1. The results thus obtained are listed in Table 2.

### Comparative Example 3

The catalyst was prepared in the same procedures as in Example 1, except that the added amount of an antimony trioxide powder was changed into 900.4 parts by mass and the cobalt nitrate was not added, in Example 1.

For the obtained catalyst, the catalyst performance test was performed in the same method as in Example 1. The results thus obtained are listed in Table 2.

### Comparative Example 4

The catalyst was prepared in the same procedures as in Example 4, except that the cobalt nitrate was not added, in Example 4.

For the obtained catalyst, the catalyst performance test was performed in the same method as in Example 1. The results thus obtained are listed in Table 2.

### Comparative Example 5

The catalyst was prepared in the same procedures as in Example 4, except that the added amount of cobalt nitrate was changed into 273.6 parts by mass in Example 4.

For the obtained catalyst, the catalyst performance test was performed in the same method as in Example 1. The results thus obtained are listed in Table 2.

### Comparative Example 6

The catalyst was prepared in the same procedures as in Example 6, except that the added amount of an antimony trioxide powder was changed into 704.6 parts by mass in Example 6.

For the obtained catalyst, the catalyst performance test was performed in the same method as in Example 1. The results thus obtained are listed in Table 2.

TABLE 1

| | | Catalyst composition (atomic ratio) | | | | | | | | | | | | | | |
|---|---|---|---|---|---|---|---|---|---|---|---|---|---|---|---|---|
| | | Fe | Sb | C | | D | | Te | Co | G | | X | Y | Z | Si | (a + f)/b |
| Example | 1 | 10 | 19 | Cu 2.5 | Ni 0.5 | W 0.5 | Mo 1.0 | 2.0 | 0.5 | P 0.2 | B 2.0 | | Ca 0.1 | Li 0.05 | 55 | 0.553 |
| | 2 | 10 | 22 | Cu 2.5 | Ni 0.5 | W 0.5 | Mo 1.0 | 2.0 | 1.8 | P 0.2 | B 2.0 | | Ca 0.1 | Li 0.05 | 55 | 0.536 |
| | 3 | 10 | 20 | Cu 2.5 | Ni 0.5 | W 0.5 | Mo 1.0 | 2.0 | 1.4 | P 0.2 | B 2.0 | | Ca 0.1 | Li 0.05 | 55 | 0.570 |
| | 4 | 10 | 21 | Cu 2.0 | Ni 1.2 | W 0.2 | Mo 1.5 | 3.0 | 1.5 | P 1.0 | B 1.0 | In 0.2 | | K 0.1 | 58 | 0.548 |
| | 5 | 10 | 21 | Cu 2.0 | Ni 1.2 | W 0.2 | Mo 1.5 | 3.0 | 1.2 | P 1.0 | B 1.0 | In 0.2 | | K 0.1 | 58 | 0.533 |
| | 6 | 10 | 22 | Cu 1.5 | Ni 2.0 | W 0.2 | Mo 1.2 | 4.0 | 2.5 | P 1.0 | | Pr 0.1 | Pb 0.05 | | 60 | 0.568 |
| | 7 | 10 | 25 | Cu 1.5 | Ni 2.0 | W 0.2 | Mo 1.2 | 4.0 | 2.5 | P 1.0 | | Pr 0.1 | Pb 0.05 | | 60 | 0.500 |
| | 8 | 10 | 21 | Cu 1.5 | Ni 2.0 | W 0.2 | Mo 1.2 | 4.0 | 2.5 | P 1.0 | | Pr 0.1 | Pb 0.05 | | 60 | 0.595 |
| Comparative | 1 | 10 | 22 | Cu 2.5 | Ni 0.5 | W 0.5 | Mo 0.5 | 2.0 | 0.5 | P 0.2 | B 2.0 | | Ca 0.1 | Li 0.05 | 55 | 0.477 |
| Examples | 2 | 10 | 19 | Cu 2.5 | Ni 0.5 | W 0.5 | Mo 0.5 | 2.0 | 2.0 | P 0.2 | B 2.0 | | Ca 0.1 | Li 0.05 | 55 | 0.632 |
| | 3 | 10 | 23 | Cu 2.5 | Ni 0.5 | W 0.5 | Mo 0.5 | 2.0 | 0 | P 0.2 | B 2.0 | | Ca 0.1 | Li 0.05 | 55 | 0.435 |
| | 4 | 10 | 21 | Cu 2.0 | Ni 1.2 | W 0.2 | Mo 1.5 | 3.0 | 0 | P 1.0 | B 1.0 | In 0.2 | | K 0.1 | 58 | 0.476 |
| | 5 | 10 | 21 | Cu 2.0 | Ni 1.2 | W 0.2 | Mo 1.5 | 3.0 | 3.5 | P 1.0 | B 1.0 | In 0.2 | | K 0.1 | 58 | 0.643 |
| | 6 | 10 | 18 | Cu 1.5 | Ni 2.0 | W 0.2 | Mo 1.2 | 4.0 | 2.5 | P 1.0 | | Pr 0.1 | Pb 0.05 | | 60 | 0.658 |

TABLE 2

| | | | Catalyst performance test | | |
|---|---|---|---|---|---|
| | | Calcination | Apparent | Propylene con- | Acryloni- |
| | | temperature | contact time | version rate | trile yield |
| | | [° C.] | [sec] | [%] | [%] |
| Examples | 1 | 795 | 2.8 | 98.5 | 82.8 |
| | 2 | 795 | 2.9 | 98.4 | 82.7 |
| | 3 | 795 | 3.1 | 98.5 | 82.5 |
| | 4 | 785 | 3.3 | 98.1 | 82.7 |
| | 5 | 785 | 2.7 | 98.2 | 82.5 |
| | 6 | 780 | 3.1 | 98.2 | 82.3 |
| | 7 | 780 | 3.2 | 98.0 | 82.2 |
| | 8 | 780 | 3.2 | 98.1 | 82.2 |
| Compar- | 1 | 795 | 3.1 | 98.1 | 81.7 |
| ative | 2 | 795 | 2.9 | 98.0 | 81.5 |
| Examples | 3 | 795 | 3.4 | 97.8 | 81.1 |
| | 4 | 785 | 3.4 | 97.7 | 81.6 |
| | 5 | 785 | 3.2 | 98.6 | 81.0 |
| | 6 | 780 | 3.5 | 98.5 | 81.3 |

As could be confirmed from Table 2, the catalysts according to Examples could have high yield of acrylonitrile as compared with Comparative Examples that did not meet the requirements of 0.50 or more and 0.60 or less of (a+f)/b.

### INDUSTRIAL APPLICABILITY

The catalyst for acrylonitrile production according to the invention can achieve high yield of acrylonitrile when producing acrylonitrile by the vapor phase contact ammoxidation of propylene, and thus can produce acrylonitrile industrially advantageously. Therefore, the catalyst is industrially very useful.

1. A catalyst represented by General Formula:
FeₐSb_{b}C_{c}D_{d}TeₑCo_{f}G_{g}XₓY_{y}Z_{z}Oₕ(SiO₂)ᵢ,
wherein
Fe is iron;
Sb is antimony;
Te is tellurium;
Co is cobalt;
C is at least one kind of element selected from the group consisting of copper and nickel;
D is at least one kind of element selected from the group consisting of molybdenum, tungsten, and vanadium;
G is at least one kind of element selected from the group consisting of phosphorous and boron;
X is at least one kind of element selected from the group consisting of tin, titanium, zirconium, niobium, tantalum, ruthenium, palladium, silver, aluminum, gallium, indium, thallium, germanium, arsenic, bismuth, lanthanum, cerium, praseodymium, neodymium, and samarium;
Y is at least one kind of element selected from the group consisting of magnesium, calcium, strontium, barium, manganese, zinc, and lead;
Z is at least one kind of element selected from the group consisting of lithium, sodium, potassium, rubidium, and cesium;
O is oxygen;
(SiO₂) represents silica;
a, b, c, d, e, f, g, x, y, z, h, and i represent the atomic ratios of respective elements, and in the case of silica, the atomic ratio of silicon;
a is 10; b is 5 to 60; c is 1 to 8; d is 0.1 to 4; e is 0.1 to 5; f is 0.1 to 4.5; g is 0.1 to 5; x is 0 to 5; y is 0 to 5; z is 0 to 2; i is 10 to 200; h is the atomic ratio of oxygen that is necessary to meet the atomic values of represent elements except silicon; and
(a+f)/b ranges from 0.50 to 0.60.

2. The catalyst according to claim 1, that comprises iron antimonate in a crystal phase.

3. A method for producing acrylonitrile comprising:
reacting propylene with molecular oxygen and ammonia in the presence of the catalyst according to claim 1.

4. A method for producing the catalyst according to claim 1 comprising:
preparing an aqueous slurry of raw materials comprising elements constituting said General Formula FeₐSb_{b}C_{c}D_{d}TeₑCo_{f}G_{g}XₓY_{y}Z_{z}Oₕ(SiO₂)ᵢ,
drying the aqueous slurry, and
calcining the dried slurry.

5. The catalyst according to claim 1, wherein C is copper and nickel.

6. The catalyst according to claim 1, wherein D is molybdenum and tungsten.
